(19) United States
(12) Patent Application Publication
(10) Pub. No.: US 2020/0388379 A1
(43) Pub. Date: Dec. 10, 2020

(54) SYSTEM AND METHOD FOR REDUCING TRAINING FOR STAFF QUALIFICATION

(71) Applicant: Trifecta Multimedia, LLC, dba Trifecta Clinical, Los Angeles, CA (US)

(72) Inventors: Erick Marquez, Los Angeles, CA (US); Andrew Chan, Los Angeles, CA (US); Kevin Beyer, Los Angeles, CA (US); Amanda Steenbergen, Indianapolis, IN (US); Brian Schilb, Indianapolis, IN (US); Grant Murakami, Honolulu, HI (US); Juan Yu, Los Angeles, CA (US); Eric Zudak, Indianapolis, IN (US); Craig Otta, Davenport, IA (US); Gabe Kwok, Los Angeles, CA (US); Daria Coltrain, Los Angeles, CA (US); Michael Demo, Indianapolis, IN (US)

(21) Appl. No.: 16/894,632

(22) Filed: Jun. 5, 2020

Related U.S. Application Data
(60) Provisional application No. 62/858,257, filed on Jun. 6, 2019.

(51) Int. Cl. G16H 40/20; G09B 5/06; G06Q 50/20; G06Q 10/10; G16H 10/20; G06Q 10/06; G16H 70/20; H04N 21/2187

(52) U.S. Cl. CPC G16H 40/20; G09B 5/065; G06Q 50/2057; G06Q 10/105; H04N 21/2187; G06Q 10/06316; G06Q 10/063112; G16H 70/20; G16H 10/20

(57) ABSTRACT

A method for reducing training requirements for clinical trial staff including receiving clinical trial information for a clinical trial, wherein the clinical trial information includes one or more clinical trial tasks, one or more training units associated with at least one of the tasks, and clinical trial protocol requirements; providing a list of the clinical trial tasks to a user for selection, wherein the user is a member of the clinical trial staff; generating a training program for the user, comprising a plurality of assigned training units, wherein each of the assigned training units are associated with the selected clinical trial tasks; providing the assigned training units from the training program to the user for completion; documenting completion of the assigned training units; and documenting confirmation that the user is qualified for the clinical trial after completion of the assigned training units, wherein confirmation is provided by a principal investigator.

### CROSS-REFERENCE TO RELATED APPLICATION

This application claims priority to, and the benefit of, U.S. provisional patent application No. 62/858,257, filed on Jun. 6, 2019, the entire disclosure of which is incorporated herein in its entirety by reference.

### BACKGROUND

### 1. Field

Embodiments of the present invention relate to a system and a method for more efficiently providing training for staff qualification.

### 2. Description of the Related Art

Various systems allow for qualifying staff before their participation in clinical studies. It is difficult, however, to efficiently administer qualification materials such that the material is not overbroad or redundant and is instead tailored to a specific staff member and his/her specific combination of responsibilities at a particular clinical research site for a given clinical study. As such, there is a need for improved methods and systems for qualifying staff.

### SUMMARY

Aspects of embodiments of the present invention relate to a system and method for staff qualification.

An embodiment of the present disclosure may provide a method for reducing training requirements for clinical trial staff including receiving clinical trial information for a clinical trial, wherein the clinical trial information includes one or more clinical trial tasks, one or more training units associated with at least one of the tasks, and clinical trial protocol requirements; providing a list of the one or more clinical trial tasks to a user for selection through a user device, wherein the user is a member of the clinical trial staff; generating a training program for the user, comprising a plurality of assigned training units, wherein each of the assigned training units are associated with the selected clinical trial tasks; providing the assigned training units from the training program to the user for completion; documenting completion, by the user, of the assigned training units; and documenting confirmation that the user is qualified for the clinical trial after completion of the assigned training units, wherein confirmation is provided by a principal investigator for the clinical trial. The one or more training units may be associated with the one or more clinical trial tasks based on a location of the clinical trial. The one or more training units may be associated with the one or more clinical trial tasks based on clinical trial protocol requirements. The method may further include including one or more training units in the training program based on a location of the clinical trial. The method may further include including one or more training units in the training program based on clinical trial protocol requirements. The method may further include receiving approval from the principal investigator before providing the assigned training units from the training program to the user for completion. The clinical trial information may include a documentation log and completion of the training units and confirmation from the principal investigator may be recorded in the documentation log. The documentation log may include training unit completion information for each of the one or more clinical trial staff members. The one or more training units may include one or more live video presentations. The method may further include removing one or more training units from the training program based on the clinical trial protocol requirements.

An embodiment of the present disclosure may provide a system for reducing training requirements for clinical trial staff including a user device configured to receive user selections and a processor with a memory device, wherein the memory device is configured to store instructions that, when executed by the processor, cause the processor to receive clinical trial information for a clinical trial, wherein the clinical trial information includes one or more clinical trial tasks, one or more training units associated with at least one of the tasks, and clinical trial protocol requirements; provide a list of the one or more clinical trial tasks to a user for selection through the user device, wherein the user is a member of the clinical trial staff; generate a training program for the user, comprising a plurality of assigned training units, wherein each of the assigned training units are associated with the selected clinical trial tasks; provide the assigned training units from the training program to the user for completion; document completion, by the user, of the assigned training units; and document confirmation that the user is qualified for the clinical trial after completion of the assigned training units, wherein confirmation is provided by a principal investigator for the clinical trial. The one or more training units may be associated with the one or more clinical trial tasks based on a location of the clinical trial. The one or more training units may be associated with the one or more clinical trial tasks based on clinical trial protocol requirements. The processor may include one or more training units in the training program based on a location of the clinical trial. The processor may include one or more training units in the training program based on clinical trial protocol requirements. The processor may receive approval from the principal investigator before providing the assigned training units from the training program to the user for completion. The clinical trial information may include a documentation log and completion of the training units and confirmation from the principal investigator may be recorded in the documentation log. The documentation log may include training unit completion information for each of the one or more clinical trial staff members. The one or more training units may include one or more live video presentations. The processor may remove one or more training units from the training program based on the clinical trial protocol requirements.

### BRIEF DESCRIPTION OF THE DRAWINGS

Example embodiments of the present invention will now be described more fully hereinafter with reference to the accompanying drawings, wherein:

FIG. 1 is a block diagram of a system for providing training in accordance with one embodiment of the present invention;

FIG. 2A-2D show an exemplary delegation of responsibilities log in accordance with an embodiment of the present invention;

FIG. 3 is a process flow chart depicting operations for processing user data to identify training content tailored to a user's specific requirements in accordance with an embodiment of the present invention;

FIG. 4 is a process flow chart for providing training in accordance with an embodiment of the present invention; and

FIG. 5 is a block diagram of an exemplary computer system in accordance with an embodiment of the present invention.

### DETAILED DESCRIPTION

Sponsors such as pharmaceutical and medical device companies conduct clinical trials (e.g., clinical trials, clinical studies, etc.) using sites (e.g., institutions, hospitals, clinics, investigative sites, clinical sites, clinical research site, clinical study site, etc.) that may identify, enroll, treat, and document clinical trial subjects according to an approved clinical study protocol. To fulfill clinical practice guidelines (e.g., International Conference on Harmonisation Good Clinical Practice Guidelines) staff generally need to review and/or receive qualification material (e.g., attend training and/or review training content such as good clinical practice training, protocol-specific training, etc.) to ensure staff perform clinical studies in accordance with approved protocols and/or guidelines.

Before a clinical trial begins, staff are generally assigned roles based on their responsibilities in the clinical trial. Staff members complete training for their assigned roles and their completion of the training is recorded to provide documentation that the staff member is qualified to perform their assigned responsibilities (e.g., tasks, duties, functions, etc.). The document may be kept at the investigative site, or is accessible from the investigative site, to allow for inspections during audits.

As discussed above, investigative sites are required to keep documentation (e.g., a delegation of responsibilities log, such as the TransCelerate Site Signature and Delegation of Responsibilities Log) that confirms each staff member is qualified to perform the tasks they are responsible for in a clinical study. Such documentation may include a list of staff members, a list of tasks that may be performed for the trial, the assignment of these tasks to the staff members, and the signature of the principal investigator of a study confirming that tasks will only be delegated to qualified staff members. Staff may not begin work until training for their assigned tasks is completed. If tasks and/or responsibilities change, the staff member completes additional training that may be necessary and the documentation is then updated to reflect the change in tasks and to confirm the associated training was completed. While the documentation is used to keep a record of staff responsibilities and confirm each staff is qualified for their respective tasks, documentation such as a delegation of responsibilities log is generally not used to determine training needs. Documentation such as a delegation of responsibilities log (DRL) is generally filled out after training is completed to confirm all staff are qualified.

The required training is generally determined based on the roles assigned to the staff members. Trainings are generally generic (e.g., live and recorded presentations given at a “lowest common denominator”) rather than tailored to the needs of the staff at a particular investigative site for their particular responsibilities at a particular clinical trial. Roles are generally broad categories that are assigned to staff and may include, for example, principal investigator, sub-investigator, and study coordinator. However, because these categories are broad, there may often be great differences, even within the same site, in the tasks assigned to staff members who are assigned to the same nominal role. Variances may be related to the country in which the site is located, requirements of the specific protocol, and/or requirements of the specific site. For example, the tasks performed by a “sub-investigator” in one country may be different from the tasks performed by a “sub-investigator” in another country.

Because roles are broad categories and training may often be assigned based on roles, the training may be formulated to be overbroad, redundant, inefficient, and/or have a “one-size-fits-all” approach because, to ensure all staff receive the necessary training for their particular clinical study, each staff member may be required to show completion of all training that could possibly be required by their role, across the range of possible tasks that could be assigned to that role. This “one-size-fits-all” or “lowest common denominator” approach to training may thus cover areas that are not relevant to a staff member's actual assigned work or tasks for a particular clinical study. For example, training provided to staff members categorized into a certain role may include topics that are not relevant to the responsibilities of every staff member who is categorized in that role. Further, such training assignments may not take into account training that may have been recently completed. For example, if a staff member works on multiple and/or consecutive studies that are related to the same disease, it may make sense to exempt the staff member from having to repeat certain training that is related to the disease. It should also be noted that, while the term “training” is generally used to refer to the qualification content and presentations assigned to staff, staff are oftentimes knowledgeable, educated, and/or licensed for their work for a clinical trial. For example, a doctor who is experienced in performing a particular physical exam, and is selected for the clinical study because of his/her experience, may still be required to complete a physical exam training for that specific type of physical exam as a part of the qualification process. Accordingly, while the term “training” may be used, the content may not be educational and may not teach a staff member anything new, but instead, that content may serve as a review of basic concepts and/or requirements in order to meet the staff member qualification requirements for the clinical study.

Some embodiments of the present invention provide more efficient training of staff members by tailoring the training to the particular tasks that will be performed by those staff members, rather than assigning training based on their assigned roles. In some embodiments, the tailoring of training includes incorporating the process used to document the qualification of staff (e.g., a delegation of responsibilities log) into the training process.

Some embodiments of the present invention may organize training programs for clinical study staff members using tasks that may be assigned to staff for the clinical study. These task assignments are also recorded in a delegation of responsibilities log (DRL). Training content may be created to train staff on one or more tasks. Information about the relationship between training content and tasks may be created and used to create training programs. After a staff member is assigned his/her tasks for a clinical study, a training program for the staff member may be created based on the assigned tasks, as well as information identifying the clinical study in which he/she is staffed (e.g., his/her name or protocol number for the clinical study). The training content may be incorporated into the training program.

By identifying the tasks assigned to staff members before training begins instead of after training is completed, and by tailoring training assignments based on the tasks that are also used in a delegation of duties log, embodiments of the present invention integrates the training process with the documentation process for staff qualification, reduces the time staff spend on completing training for clinical studies; reduces the number of training assignments; reduces the clerical work involved in administering, creating, updating, and tracking trainings; and allows training assignments to be more targeted toward specific staff responsibilities. By utilizing documentation such as a delegation of responsibilities log instead of broad categories to generate a fine-tuned training program, embodiments of the present invention enable investigative sites to complete the staff qualification process much more efficiently and start clinical trials much more quickly. This frees up time for other duties (e.g., patient care, running the clinical trial) and allows clinical trials to progress much more quickly.

Features of the inventive concept and methods of accomplishing the same may be understood more readily by reference to the following detailed description of embodiments and the accompanying drawings. Hereinafter, example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples, and will convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated.

When a certain embodiment may be implemented differently, a specific process order may be performed differently from the described order. For example, two consecutively described processes may be performed substantially at the same time or performed in an order opposite to the described order.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having meanings that are consistent with their meanings in the context of the relevant art and/or the present specification, and should not be interpreted in an idealized or overly formal sense, unless expressly so defined herein.

Aspects of embodiments of the present invention are directed to providing efficient training that is customized based on the requirements of different clinical studies, the locations of such clinical studies, and various duties of different staff members in the clinical studies. While comparative methods may identify training for a staff member based on a broad categorization of staff responsibilities in a clinical trial (e.g., role), such categories are generally overly broad because the categories group together people who perform a wide variety of different tasks. For example, two people who do not have any overlapping tasks may be grouped into the same category.

Variations in staff member responsibilities may be due to the clinical study location (e.g., the country in which the clinical study site is located follows different laws, different uses and/or meanings associated with staff roles and/or duties, etc.). Training requirements may vary among different study protocols, countries, regions, and/or states. However, despite these variances, existing methods may still group staff into the same category because they have the same title.

Oftentimes these categories are set up to capture all possible training requirements regardless of the specific tasks and requirements of a specific investigative site and the protocol for the study. These categories are not a reliable indicator of the training that is required in order for a person to be qualified as a staff member. Because staff members are assigned training based on broad categories, staff may be assigned to a wide scope of training content that oftentimes includes training that is unnecessary and/or not applicable to that staff member's duties.

As such, models for providing training to staff can be improved by customizing the training content for different individuals based on their tasks in a clinical trial.

Aspects of embodiments of the present invention are directed to efficiently building, providing, and tracking training for each staff member of a clinical site. While existing methods provide a form through which staff and training information can be documented, keeping such records is oftentimes not a part of the training process. As such, methods for organizing, providing, and tracking training for staff can be improved by integrating the documentation process with the training process.

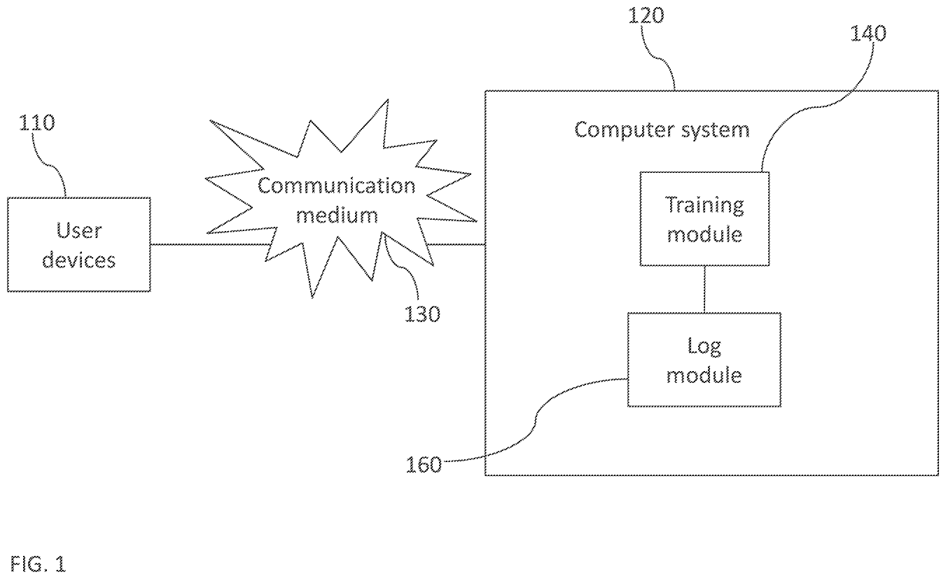

FIG. 1 is a block diagram of a system for providing training in accordance with one embodiment of the present invention. The system includes one or more user devices 110, a computer system 120, and a communication medium 130. The computer system 120 may be a clinical qualification management system. The user devices 110 may be coupled to the computer system 120 over the communication medium 130. The communication medium 130 may be a local area network (LAN), private wide area network (WAN), the Internet, or any wired or wireless network environment conventional in the art. The one or more user devices 110 may include one or more computers and/or one or more mobile devices, such as mobile phones.

The computer system 120 executes software instructions and interacts with components of the system to perform the instructions of the present invention. In one embodiment, a number of software objects or modules may reside in the computer system 120, including a training module 140 and a log module 160. Although these modules are assumed to be separate functional units, a person of skill in the art will recognize that the functionality of the modules may be combined or integrated into a single module, or further subdivided into further sub-modules, without departing from the spirit of the invention. In addition, in some embodiments, the various modules may be implemented by different physical hardware computer systems. For example, the training module 140 may be implemented in the cloud, e.g., by a server computer that may receive data over a communication medium 130 such as the Internet or other communications network, and transmit data over the communication medium 130 to the user device 110.

The training module 140 is configured to identify and provide training content for one or more users based on user data. The users may be staff members at an investigative site participating in a study. User data may include biographical information, such as name, age, address, and email address, as well as information associated with a clinical study for which the user may perform tasks, such as an identification number for the clinical study (e.g., Protocol ID, protocol number, etc.); location (e.g., country, state, province, etc.) of the investigative site for the clinical study; and a role that may be associated with the user for the clinical study (e.g., principal investigator, sub-investigator, study coordinator, etc.). User data may further include information identifying one or more tasks each of the users may perform during the course of the clinical study (e.g., maintain essential documents, obtain informed consent, perform study-related assessments, perform physical exams, etc.).

Training content may include video content, audio content, static material, and/or interactive user interfaces. Training content may include information on, for example, procedures for performing a study-related physical exam or procedures for dispensing a drug for the clinical study. The training content may include video of live trainings and/or recorded trainings. The interactive user interfaces may include, for example, expandable graphics, expandable text, quizzes, simulations, three-dimensional models, and the like.

Training content may be organized into one or more training units. Each of the training units may include different training content and may fulfill training requirements for one or more tasks. For example, a staff member may be required to complete three different training units for one task and may be required to complete one additional training unit for another task. Each of the training units may be stored and/or transferred as a file. The file size of each of the files may be limited to allow each of the files to transfer and/or be viewable through a slow communication medium (e.g. a slow internet connection) and/or a slow user device (e.g., a slow computer).

User data and/or training content may be provided to the computer system 120 via the communication medium 130 and/or from the user devices 110. User data and/or training content may be stored in the computer system 120. Alternatively, the user data and/or training content may be stored in other locations, such as the “Cloud,” where the data may be stored on a server. In one embodiment, the user data and/or training content may be provided to other sources for processing before the user data and/or training content is stored in the computer system 120 or another location. For example, the training content may be translated to a different language and/or converted to a country-specific format. In some embodiments of the present invention, the training units are delivered as one or more web pages or websites accessible through a web browser.

The computer system 120 may be configured to generate and/or update one or more certificates for a user after the user completes one or more training units. The certificate may display all training units that have been completed by the user. For each training unit that is completed, the certificate may identify the associated task. For example, various training units that are completed for one task may be organized into one section for the task.

The log module 160 is configured to generate and/or update one or more delegation of responsibilities logs for clinical studies. A delegation of responsibilities log may also be referred to as a delegation of duties log. A delegation of responsibilities log may be used to fulfill clinical practice guidelines (e.g., International Conference on Harmonisation Good Clinical Practice Guidelines), Such guidelines may require a document that provides a list of qualified and trained staff who may perform tasks at an investigative site in conduct of a clinical study; documents roles and/or responsibilities assigned to staff of the clinical study: and/or documents signatures and/or initials of individuals who are authorized to make entries and/or corrections on the document.

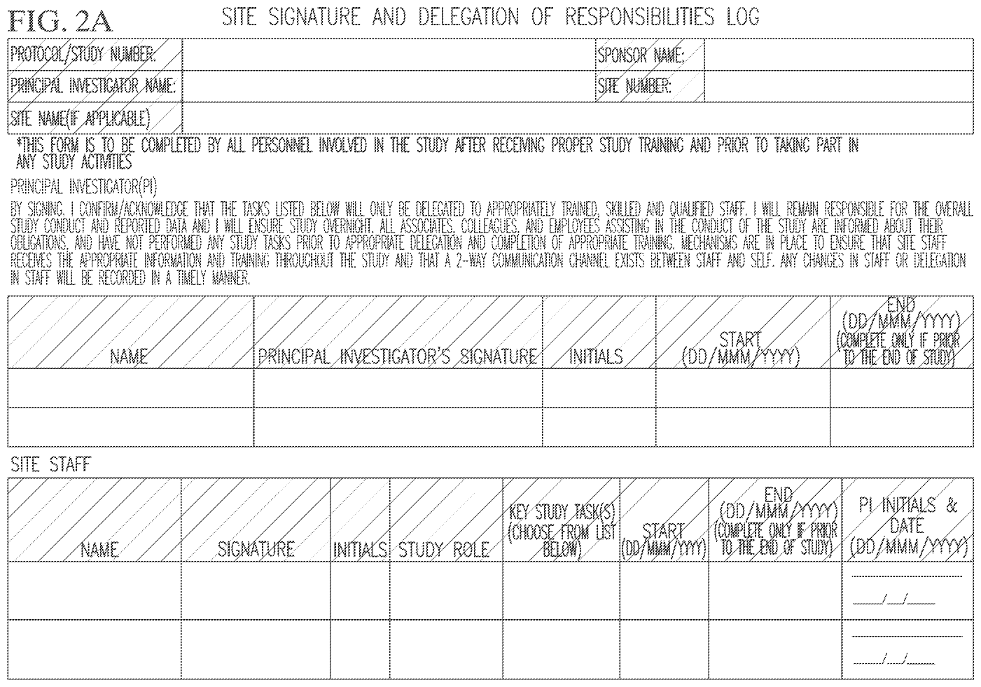

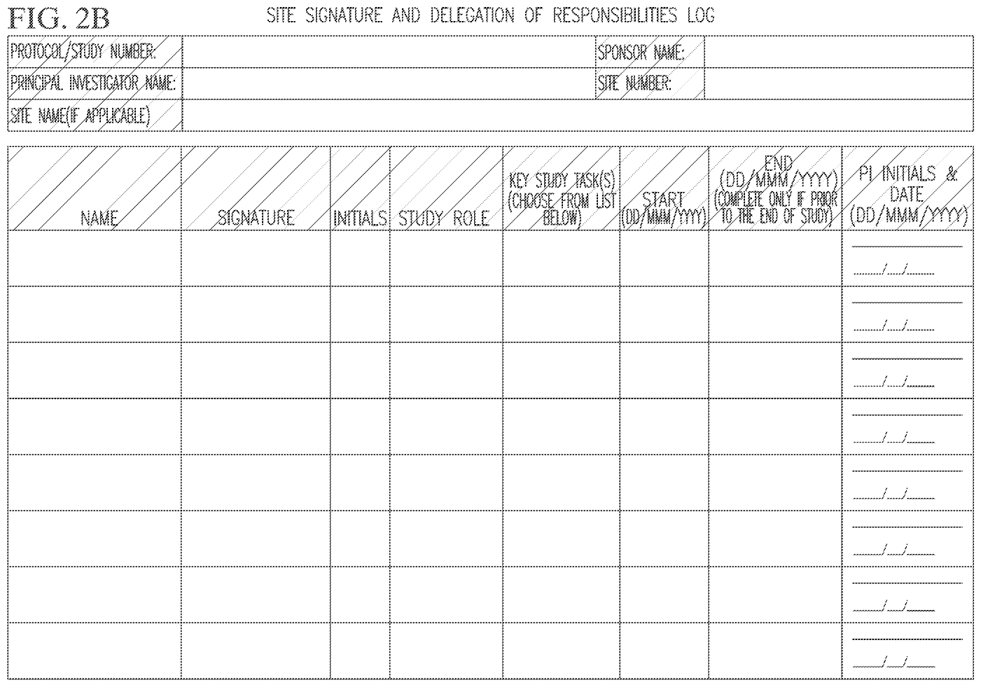

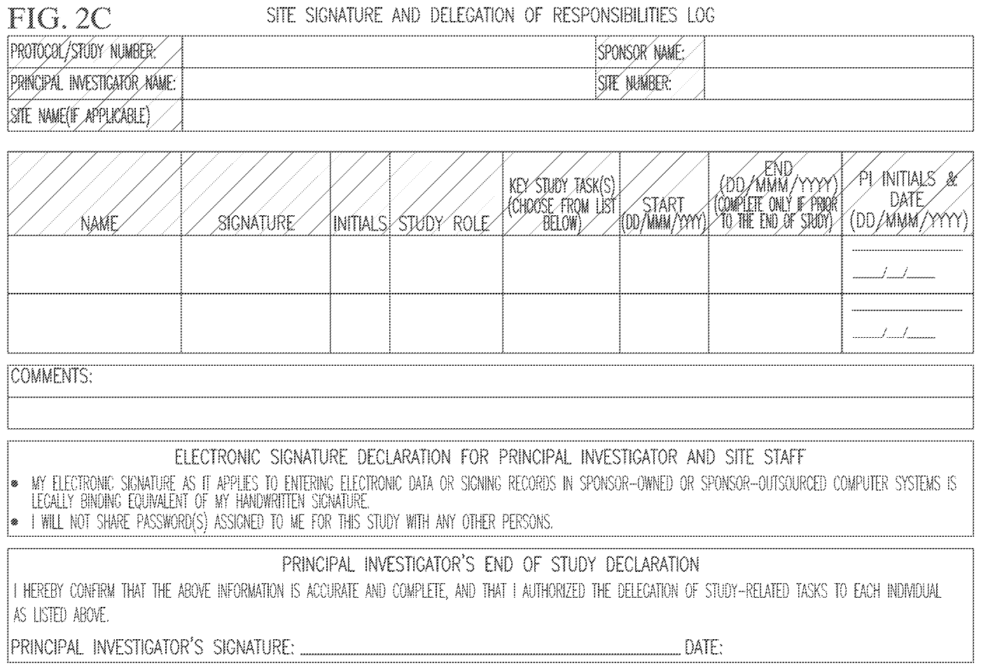

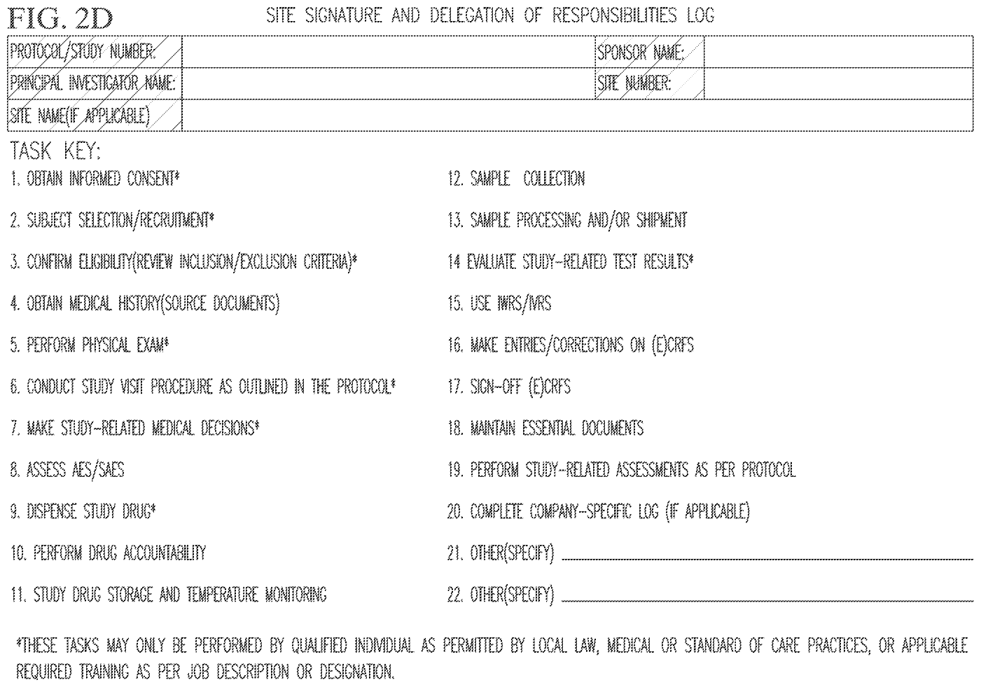

FIG. 2A-2D show an exemplary delegation of responsibilities log (DRL) in accordance with an embodiment of the present invention. As discussed, the DRL may be used to document the qualification of staff members for their work in clinical studies. A system administrator for the computer system 120 and/or an operator involved with operating the clinical trial may provide the DRL to the computer system 120 with blank fields that can be filled with staff member information. When a staff member identifies for the computer system 120 the tasks that are assigned to him/her, the computer system 120 generates a training program for the staff member, where the training program includes one or more training units, and the computer system 120 may also record the tasks that are assigned to the staff member in the DRL. When the staff member completes the training program, the computer system 120 records the completion of the training. As needed, the computer system 120 generates a completed DRL based on the information recorded regarding the tasks assigned to each staff member and whether the staff member has been qualified to perform the tasks (e.g., by having completed the required training)

In addition, the computer system 120 may determine and/or organize training programs for staff members based on tasks that may be associated with a particular DRL. For example, the DRL from two different clinical studies may have different task names for the same work and/or a task name may be defined differently in different clinical studies and thus require different training. Information about training requirements for tasks that are associated with each DRL may be provided to the computer system 120 by a system administrator for the computer system 120 and/or an operator involved with operating the clinical trial.

The DRL may include information identifying the clinical study, information about the staff members of the clinical study and their responsibilities, and/or information about the training completed by staff members.

For example, the DRL may include a field for the protocol or study number; a field for the name of a principal investigator; a field for the name of the clinical study site; a field for the sponsor name; and/or a field for a site number.

The DRL may include fields for information associated with one or more principal investigators of a clinical study. Such fields may include fields for the name of one or more principal investigators of the clinical study; fields for the signature of the corresponding principal investigator (e.g., an electronic signature) and date and/or time information indicating when the signature was provided; fields for the initials of the corresponding principal investigator and date and/or time information indicating when the initials were provided; fields for a start date; and fields for an end date.

The DRL may include fields for information associated with one or more staff members of a clinical study. Such fields may include fields for the name of one or more staff members; fields for the signature of the corresponding staff member (e.g., an electronic signature) and date and/or time information indicating when the signature was provided; fields for the initials of the corresponding staff member and date and/or time information indicating when the initials were provided; fields for the role of the staff member; fields for tasks that may be performed by the staff member for the clinical study (e.g., tasks that are listed in the task key); fields for a start date; fields for an end date; and/or fields for the initials of the principal investigator of the study (e.g., an electronic signature) and date and/or time information indicating when the initials were provided.

The DRL may include a declaration section, where a principal investigator for the study may provide a signature to declare that the information on the DRL is accurate and complete and that the principal investigator authorized the delegation of tasks as provided by the DRL. The DRL may also include a field where comments may be added.

The DRL may include the task key. The task key may include a list of tasks that may be performed for the clinical trial and each task may be represented by a number. The fields for tasks on the DRL may be filled using the numbers in the task key.

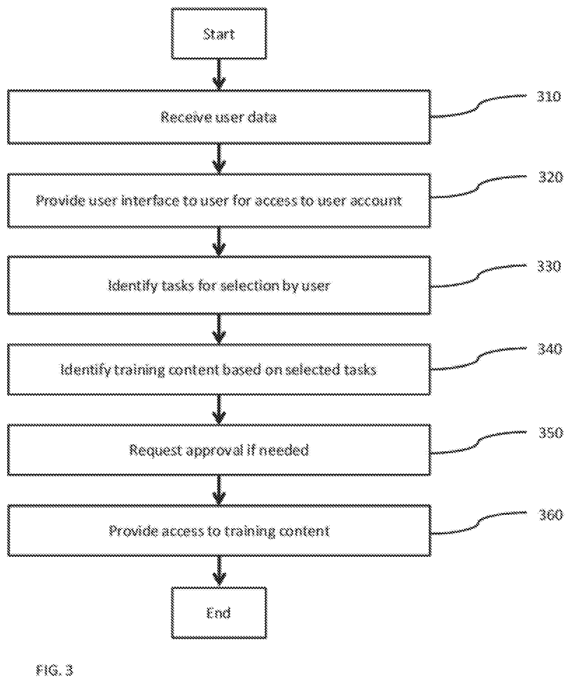

FIG. 3 is a process flow chart depicting the computer system 120 operations for processing user data to identify training content tailored to a user's specific requirements in accordance with an embodiment of the present invention. The process starts, and in operation 310, the computer system 120 receives user data. A user may be a staff member who will perform assigned tasks related to a clinical study. The user data may include user names, email addresses, and/or unique identifiers associated with each of the users. In some embodiments, the computer system 120 may generate a unique identifier for each of the users. The user data may also include information related to one or more clinical studies for which the users may perform tasks. For example, the user data may include protocol identification number for each of the clinical studies, the location of each of the investigative sites, and information identifying the role each of the users may have for the corresponding clinical studies. In some embodiments, the users may be assigned to perform one or more tasks for a plurality of clinical studies. Accordingly, the users may be associated with multiple sets of user information, where each set may include the protocol identification number, the location, and role information associated with the user for the corresponding clinical study. The user data may be transmitted to the computer system 120 from one or more user devices 110 and the user data may be transmitted to the computer system 120 via the communication medium 130. The computer system 120 may store the user data after the user data is received.

The computer system 120 may also receive information indicating whether approval is needed before each of the users have access to the training units. The users may have different access rights for different clinical studies. For example, a user may need to be approved before the user can access training for one clinical study but the user may not need to be approved before the user can access training for another clinical study. The computer system 120 may also receive information identifying one or more tasks that are associated with each of the clinical studies. The information concerning approvals and/or tasks may be transmitted to the computer system 120 from the one or more user devices 110 and the information may be transmitted to the computer system 120 via the communication medium 130. The computer system 120 may store the information after the information is received. Alternatively, the information concerning approvals and/or tasks may be stored in other locations, such as the “Cloud,” where the data may be stored on a server.

In the embodiment shown in FIG. 3, in operation 320, the computer system 120 provides a user interface to a user device 110, through which a user can access a user account that is unique to the user. The user account may be associated with the user data received by the computer system 120 for the corresponding user. The user account may further be associated with login information (e.g., user name and password) that may be used to access the user account. In some embodiments, the user may be associated with a plurality of protocol identification numbers (e.g., the user may be a staff member for multiple clinical studies), in which case the computer system 120 may provide through the user interface an option to select a protocol identification number. The user may select the protocol identification number corresponding to the clinical study for which the user desires to complete training.

In operation 330, the training module 140 identifies one or more tasks associated with a clinical study based on the protocol identification selected by the user and/or the identity of the user. Tasks may include obtaining informed consent, subject selection/recruitment, confirming eligibility, obtaining medical history, performing physical exam, conducting study visit procedure as outlined in protocol, making study-related medical decisions, assessing adverse events (AEs)/serious adverse events (SAEs), dispensing study drugs, performing drug accountability, studying drug storage and temperature monitoring, sample collection, sample processing and/or shipment, evaluating study-related test results, using interactive web response system (IWRS)/interactive voice response system (IVRS), making entries/corrections on electronic case report forms ((e)CRFs), signing off (e)CRFs, maintaining essential documents, performing study-related assessments as per protocol, and completing company-specific log. The one or more identified tasks may be specific to the location of the clinical study. For example, the identified task may be a task used by some clinical trials located in the United States but the identified task may not meet the rules and guidelines of a different country and thus the task may not be used by a clinical trial in another country. The one or more tasks may be from a list of tasks that is associated with the clinical study. For example, data for a clinical study may be provided from a user device 110 to the computer system 120 in a format that associates a clinical study with one or more tasks (e.g., a delegation of responsibilities log for the clinical study). The data may be provided by a system administrator for the computer system 120 and/or an operator involved with operating the clinical trial. The one or more identified tasks may be presented to the user for selection from a user interface. Such tasks may be duties the user may be responsible for in the corresponding clinical trial. If the user later selects an option to make another selection of tasks, the process may return to operation 330.

In operation 340, the training module 140 identifies one or more training units associated with each of the tasks selected by the user. The training module 140 may also identify one or more training units associated with other user data, such as protocol identification number and/or location of the corresponding clinical study.

The computer system 120 identifies overlaps in training such that a user will only be required to complete a training unit once even when the training unit is required by multiple tasks assigned to the user. The computer system 120 may also identify overlaps in among different protocols and/or different clinical study sites. The computer system 120 may analyze information that is associated with the user from multiple protocols and/or multiple clinical studies to identify overlaps in training content. Thus, the resulting set of training units assigned to a user does not include duplicates (e.g., duplicates are removed or, equivalently, training units that are already added to the set of training units for the user are not added again).

In some optional embodiments, in operation 350, the training module 140 may generate and deliver a message to another user requesting an approval before the user who is logged into the system is given access to the training units. The other user may be a user with special privileges and/or administrative rights (e.g., the principal investigator of a clinical trial). However, if approval information for the user indicates approval is not needed before the user has access to the corresponding training units, the process continues to operation 360 without generating or delivering a message requesting approval and without receiving information indicating that access by the user has been approved. If approval information indicates approval is needed before the user has access to the corresponding training units, the process continues to operation 360 after information is received indicating that access by the user has been approved.

In operation 360, the training module 140 provides the user with access to the training units. The computer system 120 may deliver the training units to the user device via the communication medium 130. The operation may end after operation 360 or the operation may return to operation 330 if the user selects an option to make another selection of tasks. Embodiments of the present invention include any combination of the optional steps.

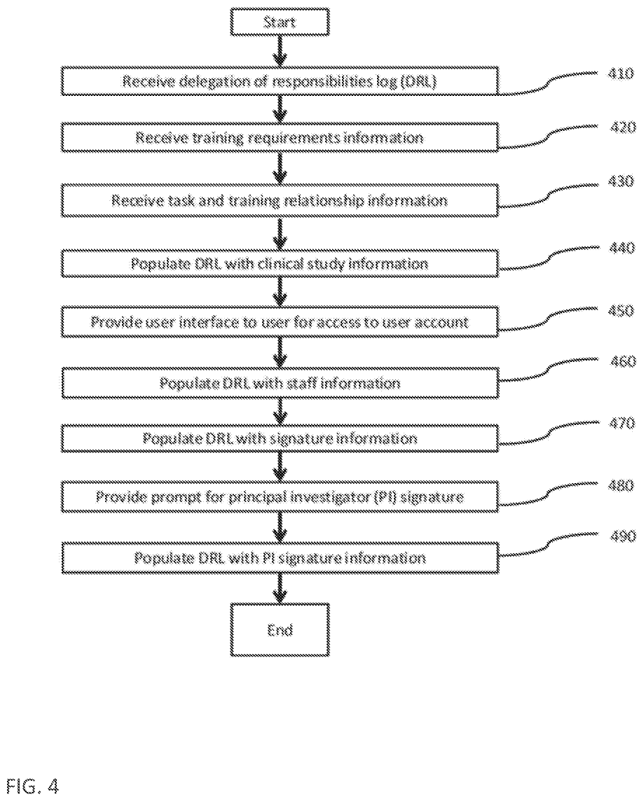

FIG. 4 is a flow chart of a method of providing training, according to an embodiment of the present disclosure. At 410, the computer system 120 receives a delegation of responsibilities log (DRL) for a clinical trial. While the DRL will generally include fields into which information may be inputted, the DRL may be provided to the computer system 120 with one or more fields pre-populated (e.g., staff member names may be pre-populated while the fields for the tasks assigned to staff may be blank when the computer system 120 receives the DRL). The DRL may be provided by a system administrator for the computer system 120 and/or an operator involved with operating the clinical trial. The task key of the DRL may include a list of tasks that is customized based on the needs of the clinical trial.

In some optional embodiments, at 420, the computer system 120 may receive information about training requirements associated with each of the tasks included in the task key of the DRL. For example, there may be requirements that are associated with the location of the clinical study and/or requirements that are associated with the study protocol. Information about training requirements may be provided by a system administrator for the computer system 120 and/or an operator involved with operating the clinical trial.

At 430, the computer system 120 receives information about one or more relationships between tasks and training units. In some embodiments, the information is used to associate one or more training units to each task. Such information may be used by the computer system 120 to determine what training units need to be completed for each of the tasks.

In some optional embodiments, at 440, the log module 160 may populate the DRL with information such as a protocol and/or study number and a sponsor name for the clinical study. This information may be provided by a system administrator for the computer system 120 and/or an operator involved with operating the clinical trial. In some embodiment, this information may be provided with the DRL at operation 410.

At 450, the computer system 120 provides a user interface to a user device 110, through which a user may access a user account that is unique to the user, select a protocol number for the clinical study for which the user desires to complete training, and/or select the one or more tasks the user may perform for the clinical study. As discussed with respect to FIG. 3, the training module 140 may determine what tasks are provided to the user for selection based on the user's identity and/or the protocol selected by the user.

At 460, after the user selects one or more tasks, the log module 160 may populate the DRL with information such as the name of the principal investigator for the clinical study, the site number of the clinical study, the site name of the clinical study, the name of the staff member of the clinical study, the role of the staff member in the clinical study, and/or the tasks selected by the staff member. After the user selects the one or more tasks, the training module 140 provides training content to the user. Provision of the training content may occur based on the need for approvals before access is granted to the user. If the user later selects an option to make another selection of tasks, the method may return to operation 460.

If a training unit was previously completed (e.g., for a different study protocol), a user may not need to complete the training unit again. For example, a training unit that is eligible for cross-study exemption, cross-sponsor exemption, and/or is completed within an expiration period may not need to be completed for multiple clinical studies. If multiple tasks share some training units, those training units would only need to be completed once by the user and completing those training units once would progress the user toward qualification for both tasks. Similarly, if multiple study protocols share some training units, completing those training units once would progress the user toward qualification for both protocols. An exemption can be provided when the training provides, for example, background information about a particular disease and multiple studies are investigating the same disease. An exemption can also be provided when the training provides, for example, information on a drug that is being tested in multiple studies, even when there are variations in the study protocol, such as drug dosage. This is one way in which embodiments of the present invention improve the efficiency of training—by detecting redundant training.

If an expiration date is associated with a training unit that has been completed by a user, and the expiration date is approaching, the computer system 120 may provide a notification to the user, notifying the user that the training unit needs to be completed again. The notification may be in the form of a pop-up notification that appears after the user logs into his/her user account and/or the notification may be an email that is delivered to the user even when the user is not logged into his/her user account.

In some optional embodiments, at 470, after the user completes all training units for each of the selected tasks, the log module 160 may provide a prompt to the user that may allow the user to provide a signature (e.g., an electronic signature) and/or initials to confirm the user has completed all training units for the selected tasks.

The log module 160 may populate the DRL with the signature and/or initials provided by the user, as well as date and/or time information indicating when the signature and/or initials were provided. The log module 160 may also populate the DRL with a start date (the start date may be the date the user provides a signature and/or initials for DRL).

In some optional embodiments, at 480, a notification may be provided to a user who is the principal investigator of the clinical study. The notification may request the user to confirm and/or acknowledge the information that has been added to the DRL. For example, the principal investigator may need to confirm that the staff member shown on the DRL and the corresponding tasks listed for the staff member are correct and that the staff member has completed training for each of the listed tasks. The principal investigator may confirm and/or acknowledge the information on the DRL is correct by providing the principal investigator's initials to the computer system 120. The principal investigator may provide initials through a user device 110.

At 490, after the computer system 120 receives the initials from the principal investigator, the log module 160 may populate the DRL with the initials provided by the principal investigator, as well as date and/or time information indicating when the initials were provided. The method may return to 460 when the user selects more tasks from the user interface and/or when another user logs into a user account that is unique to the other user and makes task selections that are relevant to the other user's duties for a clinical trial. Embodiments of the present invention include any combination of the optional steps.

Accordingly, aspects of embodiments of the present invention relate to systems and methods for improving the efficiency of providing training for qualifying staff members of an investigative site for performing a clinical research study. Some aspects of embodiments of the present invention relate to receiving a list of one or more assigned tasks for each staff member of the investigative site and generating a customized training program for the staff member, where the customized training program may include one or more training modules. Embodiments of the present disclosure also relate to automatically maintaining and generating a delegation of responsibilities log (DRL) for an investigative site by tracking which tasks are assigned to which staff members and updating the DRL when staff members complete the training module program to qualify the staff members for their assigned tasks. By generating a customized training program based on the tasks assigned to staff members, the training effort is made more efficient compared to systems in which staff members are assigned training based on their given roles, because training based on roles generally includes training on all tasks that could possibly be assigned to the role.

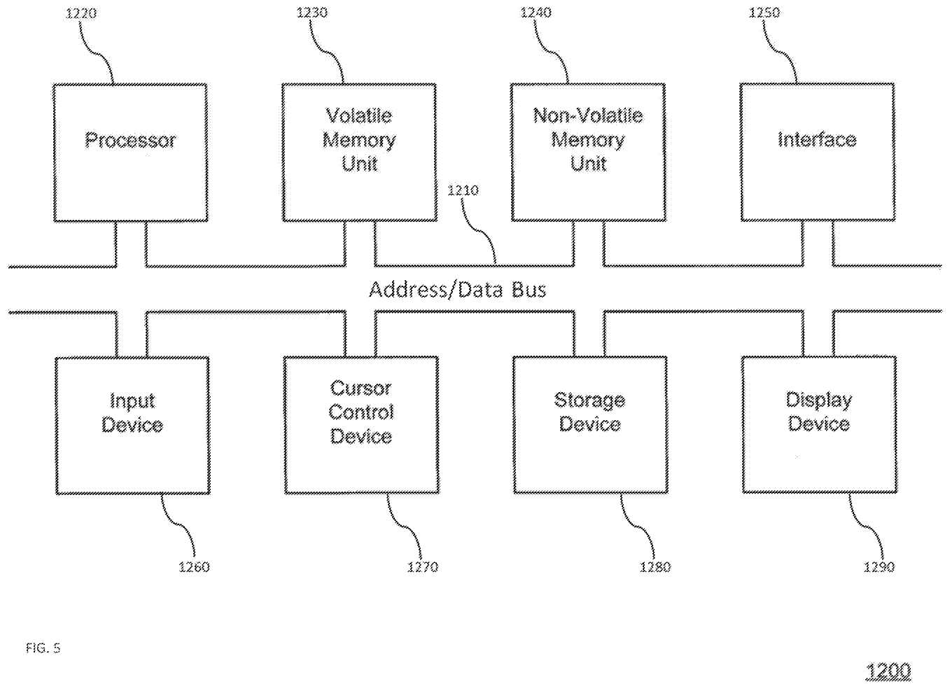

FIG. 5 is a block diagram of an exemplary computer system 1200 in accordance with an embodiment, which, in some embodiments of the present invention, may be used to implement the computer system 120 described above. Exemplary computer system 1200 is configured to perform calculations, processes, operations, and/or functions associated with a program or algorithm. In one embodiment, certain processes and operations discussed herein are realized as a series of instructions (e.g., software program) that reside within computer readable memory units and are executed by one or more processors of exemplary computer system 1200. When executed, the instructions cause exemplary computer system 1200 to perform specific actions and exhibit specific behavior, such as described herein.

Exemplary computer system 1200 may include an address/data bus 1210 that is configured to communicate information. Additionally, one or more data processing units, such as processor 1220, are coupled with address/data bus 1210. Processor 1220 is configured to process information and instructions. In an embodiment, processor 1220 is a microprocessor. Alternatively, processor 1220 may be a different type of processor such as a parallel processor or a field programmable gate array.

Exemplary computer system 1200 is configured to utilize one or more data storage units. Exemplary computer system 1200 may include a volatile memory unit 1230 (e.g., random access memory (“RAM”), static RAM, dynamic RAM, etc.) coupled with address/data bus 1210, wherein volatile memory unit 1230 is configured to store information and instructions for processor 1220. Exemplary computer system 1200 further may include a non-volatile memory unit 1240 (e.g., read-only memory (“ROM”), programmable ROM (“PROM”), erasable programmable ROM (“EPROM”), electrically erasable programmable ROM (“EEPROM”), flash memory, etc.) coupled with address/data bus 1210, wherein non-volatile memory unit 1240 is configured to store static information and instructions for processor 1220. Alternatively, exemplary computer system 1200 may execute instructions retrieved from an online data storage unit, such as in “Cloud” computing. In an embodiment, exemplary computer system 1200 also may include one or more interfaces, such as interface 1250, coupled with address/data bus 1210. The one or more interfaces are configured to enable exemplary computer system 1200 to interface with other electronic devices and computer systems. The communication interfaces implemented by the one or more interfaces may include wireline (e.g., serial cables, modems, network adaptors, etc.) and/or wireless (e.g., wireless modems, wireless network adaptors, etc.) communication technology.

In one embodiment, exemplar computer system 1200 may include an input device 1260 coupled with address/data bus 1210, wherein input device 1260 is configured to communicate information and command selections to processor 1220. In accordance with one embodiment, input device 1260 is an alphanumeric input device, such as a keyboard, that may include alphanumeric and/or function keys. Alternatively, input device 1260 may be an input device other than an alphanumeric input device. In an embodiment, exemplar computer system 1200 may include a cursor control device 1270 coupled with address/data bus 1210, wherein cursor control device 1270 is configured to communicate user input information and/or command selections to processor 1220. In an embodiment, cursor control device 1270 is implemented using a device such as a mouse, a track-ball, a track-pad, an optical tracking device, or a touch screen. The foregoing notwithstanding, in an embodiment, cursor control device 1270 is directed and/or activated via input from input device 1260, such as in response to the use of special keys and key sequence commands associated with input device 1260. In an alternative embodiment, cursor control device 1270 is configured to be directed or guided by voice commands.

In an embodiment, exemplary computer system 1200 further may include one or more optional computer usable data storage devices, such as storage device 1280, coupled with address/data bus 1210. Storage device 1280 is configured to store information and/or computer executable instructions. In one embodiment, storage device 1280 is a storage device such as a magnetic or optical disk drive (e.g., hard disk drive (“HDD”), floppy diskette, compact disk read only memory (“CD-ROM”), digital versatile disk (“DVD”)). Pursuant to one embodiment, a display device 1290 is coupled with address/data bus 1210, wherein display device 1290 is configured to display video and/or graphics. In an embodiment, display device 1290 may include a cathode ray tube (“CRT”), liquid crystal display (“LCD”), field emission display (“FED”), plasma display, or any other display device suitable for displaying video and/or graphic images and alphanumeric characters recognizable to a user.

Exemplary computer system 1200 is presented herein as an exemplary computing environment in accordance with an embodiment. However, exemplary computer system 1200 is not strictly limited to being a computer system. For example, an embodiment provides that exemplary computer system 1200 represents a type of data processing analysis that may be used in accordance with various embodiments described herein. Moreover, other computing systems may also be implemented. Indeed, the spirit and scope of the present technology is not limited to any single data processing environment. Thus, in an embodiment, one or more operations of various embodiments of the present technology are controlled or implemented using computer-executable instructions, such as program modules, being executed by a computer. In one exemplary implementation, such program modules include routines, programs, objects, components, and/or data structures that are configured to perform particular tasks or implement particular abstract data types. In addition, an embodiment provides that one or more aspects of the present technology are implemented by utilizing one or more distributed computing environments, such as where tasks are performed by remote processing devices that are linked through a communications network, or such as where various program modules are located in both local and remote computer-storage media including memory-storage devices.

Embodiments are provided to disclose the invention to those skilled in the art to which the invention pertains, and the scope of the invention should be understood by the claims of the invention. Accordingly, it will be understood by those of skill in the art that various changes in form and details may be made without departing from the spirit and scope of the invention as set forth in the following claims. Therefore, the technical range of the present invention is not limited to the detailed description of the specification but defined by the range of any claims and their functional equivalents.

What is claimed is:

1. A method for reducing training requirements for clinical trial staff, comprising:
receiving, by a processor, clinical trial information for a clinical trial, wherein the clinical trial information includes one or more clinical trial tasks, one or more training units associated with at least one of the tasks, and clinical trial protocol requirements;
providing, by the processor, a list of the one or more clinical trial tasks to a user for selection through a user device, wherein the user is a member of the clinical trial staff;
generating, by the processor, a training program for the user, comprising a plurality of assigned training units, wherein each of the assigned training units are associated with the selected clinical trial tasks;
providing, by the processor, the assigned training units from the training program to the user for completion;
documenting, by the processor, completion, by the user, of the assigned training units; and
documenting, by the processor, confirmation that the user is qualified for the clinical trial after completion of the assigned training units, wherein confirmation is provided by a principal investigator for the clinical trial.

2. The method of claim 1, wherein the one or more training units are associated with the one or more clinical trial tasks based on a location of the clinical trial.

3. The method of claim 1, wherein the one or more training units are associated with the one or more clinical trial tasks based on clinical trial protocol requirements.

4. The method of claim 1, further comprising including, by the processor, one or more training units in the training program based on a location of the clinical trial.

5. The method of claim 1, further comprising including, by the processor, one or more training units in the training program based on clinical trial protocol requirements.

6. The method of claim 1, further comprising receiving, by the processor, approval from the principal investigator before providing the assigned training units from the training program to the user for completion.

7. The method of claim 1, wherein the clinical trial information includes a documentation log, and
wherein completion of the training units and confirmation from the principal investigator are recorded in the documentation log.

8. The method of claim 7, wherein the documentation log includes training unit completion information for each of the one or more clinical trial staff members.

9. The method of claim 1, wherein the one or more training units include one or more live video presentations.

10. The method of claim 1, further comprising removing, by the processor, one or more training units from the training program based on the clinical trial protocol requirements.

11. A system for reducing training requirements for clinical trial staff, comprising:
a user device configured to receive user selections; and
a processor with a memory device, wherein the memory device is configured to store instructions that, when executed by the processor, cause the processor to:
receive clinical trial information for a clinical trial, wherein the clinical trial information includes one or more clinical trial tasks, one or more training units associated with at least one of the tasks, and clinical trial protocol requirements;
provide a list of the one or more clinical trial tasks to a user for selection through the user device, wherein the user is a member of the clinical trial staff;
generate a training program for the user, comprising a plurality of assigned training units, wherein each of the assigned training units are associated with the selected clinical trial tasks;
provide the assigned training units from the training program to the user for completion;
document completion, by the user, of the assigned training units; and
document confirmation that the user is qualified for the clinical trial after completion of the assigned training units, wherein confirmation is provided by a principal investigator for the clinical trial.

12. The system of claim 11, wherein the one or more training units are associated with the one or more clinical trial tasks based on a location of the clinical trial.

13. The method of claim 11, wherein the one or more training units are associated with the one or more clinical trial tasks based on clinical trial protocol requirements.

14. The method of claim 11, wherein the processor includes one or more training units in the training program based on a location of the clinical trial.

15. The method of claim 11, wherein the processor includes one or more training units in the training program based on clinical trial protocol requirements.

16. The method of claim 11, wherein the processor receives approval from the principal investigator before providing the assigned training units from the training program to the user for completion.

17. The method of claim 11, wherein the clinical trial information includes a documentation log, and
wherein completion of the training units and confirmation from the principal investigator are recorded in the documentation log.

18. The method of claim 17, wherein the documentation log includes training unit completion information for each of the one or more clinical trial staff members.

19. The method of claim 11, wherein the one or more training units include one or more live video presentations.

20. The method of claim 11, wherein the processor removes one or more training units from the training program based on the clinical trial protocol requirements.
